(19) United States
(12) Patent Application Publication
(10) Pub. No.: US 2022/0160454 A1
(43) Pub. Date: May 26, 2022

(54) METHOD FOR OPERATING AN AUGMENTED REALITY OBSERVATION SYSTEM IN A SURGICAL APPLICATION, AND AUGMENTED REALITY OBSERVATION SYSTEM FOR A SURGICAL APPLICATION

(71) Applicant: Carl Zeiss Meditec AG, Jena (DE)

(72) Inventors: Stefan SAUR, Aalen (DE); Christoph HAUGER, Aalen (DE); Christoph SCHAEFF, Aalen (DE)

(21) Appl. No.: 17/529,041

(22) Filed: Nov. 17, 2021

(30) Foreign Application Priority Data
Nov. 25, 2020 (DE) 10 2020 214 822.8

(51) Int. Cl. A61B 90/00; A61B 90/30; G06F 3/01; G02B 27/00; G02B 27/01; G06T 19/00

(52) U.S. Cl. CPC A61B 90/36; A61B 90/30; G06F 3/013; G02B 27/0093; G02B 27/0101; G02B 27/0179; G02B 2027/0178; G06T 19/006; A61B 2090/365; A61B 2090/368; G02B 2027/0118; G02B 2027/0187; G02B 27/017

(57) ABSTRACT

The invention relates to a method for operating an augmented reality observation system in a surgical application, wherein a viewing direction of a user is registered by means of a viewing direction sensor system of an AR observation apparatus, wherein the registered viewing direction is evaluated by means of a control device, and wherein at least one property of at least one controllable light source in the environment is altered by means of the control device by means of a control signal on the basis of the registered viewing direction. Further, the invention relates to an augmented reality observation system for a surgical application.

### CROSS-REFERENCE TO RELATED APPLICATIONS

This application claims foreign priority under 35 U.S.C. § 119 to and the benefit of German Patent Application No. 10 2020 214 822.8, filed on Nov. 25, 2020, which is incorporated herein by reference in its entirety.

### FIELD OF THE INVENTION

The invention relates to a method for operating an augmented reality observation system in a surgical application, and to an augmented reality observation system for a surgical application.

### BACKGROUND

When applications of augmented reality, for example with the aid of augmented reality glasses (AR glasses), are used in the operating room, information is provided to a user in different ways: augmented content, such as, e.g., vessel diameters projected onto a situs, tumor information projected onto the situs, etc., virtual windows with additional information regarding the patient (e.g., pulse, ECG, computed tomography data, magnetic resonance data, ultrasound data, . . . ) and/or three-dimensional renderings of imaging methods with anatomical information. Further, it is also possible to observe real content through the AR glasses or past the AR glasses, for example physical devices or monitors, an environment or the situs. Lastly, it is also possible to look through the AR glasses at a real screen or a real device with virtual information thereon which has been enriched by means of the AR glasses.

A problem occurring in the process is that of there being a change in image quality or image properties of an image which is registrable by a user and which is of a real and/or augmented environment when there is a change between different sources of information, and this may lead to an impediment to a smooth workflow since the user must first adapt to the altered image quality or the altered image properties. In particular, the eyes of the user must adapt to an altered image quality or altered image properties, requiring an adaptation time.

US 2017/0256095 A1 has disclosed a blocking screen for an augmented reality application. To control the visibility of augmentations in different situations, the blocking screen is arranged such that a brightness of a real scene can be attenuated. The blocking screen attenuates the light more strongly at a few points under program control and offers a region where the augmentation information can be better displayed. An extent of the attenuation overall or for certain parts of the blocking screen can be altered in order to take account of the brightness and/or disturbances in the real scene.

CN 110 262 041 A has disclosed an augmented reality display method and an apparatus. The method comprises steps in which the light of a virtual image and the light reflected by an external physical object are directed at a human eye by way of a display module; a color sensor is used to collect the color information of the light reflected by the external physical object and to transmit the color information to a controller. The light reflected by the external physical object is filtered by a filter apparatus. The color information of the virtual image is registered from an image source by way of the controller and the wavelength range of the light filtered by the filter apparatus is controlled in accordance with the color information of the light reflected by the external physical object and the color information of the virtual image in order to control the color of the light reflected by the external physical object in the human eye. The contrast between the image color of the physical object seen by the human eye and the color of the virtual image is controlled as required in order to obtain a better visual effect.

### SUMMARY OF THE INVENTION

The invention is based on the object of developing a method for operating an augmented reality observation system in a surgical application and an augmented reality observation system for a surgical application, in which, in particular, disturbances when registering the environment and during the workflow of the user can be avoided or prevented to a better extent.

According to the invention, the object is achieved by a method having the features of patent claim 1 and an augmented reality observation system having the features of patent claim 10. Advantageous configurations of the invention emerge from the dependent claims.

It is one of the basic ideas of the invention to alter at least one property of a controllable light source in the environment, in which an AR observation system is used, on the basis of a registered viewing direction of a user. In this case, on the basis of the registered viewing direction should mean that, in particular, the registered viewing direction is used to determine whether or not the at least one property of the at least one controllable light source is altered. In particular, this is based on the concept of properties of controllable light sources in the environment being able to be adapted on the basis of the viewing direction in such a way that the light sources do not cause disturbances when registering the environment and in the workflow of the user. The viewing direction of the user is registered by means of a viewing direction sensor system of an AR observation apparatus of the AR observation system. A control device of the AR observation system evaluates the registered viewing direction. Based on an evaluation result, the control device generates control signals for the at least one controllable light source in order to alter at least one property of the at least one light source on the basis of the registered viewing direction. In particular, the invention facilitates a dynamic change of properties of controllable light sources on the basis of the registered viewing direction. As a result, the controllable light sources can be controlled in such a way that disturbances when registering the environment and in the workflow of the user can be avoided or at least reduced.

In particular, a method for operating an augmented reality observation system in a surgical application is provided, wherein a viewing direction of a user is registered by means of a viewing direction sensor system of an AR observation apparatus, wherein the registered viewing direction is evaluated by means of a control device, and wherein at least one property of at least one controllable light source in the environment is altered by means of the control device by means of a control signal on the basis of the registered viewing direction.

Further, an augmented reality observation system for a surgical application, in particular, is created, comprising an augmented reality observation apparatus with a viewing direction sensor system, wherein the viewing direction sensor system is configured to register a viewing direction of a user of the AR observation apparatus, and a control device, wherein the control device is configured to evaluate the registered viewing direction and to alter at least one property of at least one controllable light source in the environment on the basis of the registered viewing direction by means of a control signal.

The method and the augmented reality (AR) observation system are used in a surgical application. In particular, such a surgical application can comprise the use of a medical surgical system, for example a surgical microscope or any other (robotic) visualization system. In particular, the method and the augmented reality (AR) observation system can be configured to be used with a medical surgical system, for example with a surgical microscope. The augmented reality (AR) observation system can also be part of, or comprise, a medical surgical system, for example a surgical microscope.

In particular, an augmented reality (AR) observation apparatus is a pair of augmented reality (AR) glasses, which a user can wear on the head. In particular, an AR observation apparatus comprises a screen, through which an environment can be registered directly and on which additional content can be projected, said additional content being able to be registered by the user together with and/or superimposed on the real environment. Content observed by the user through the AR observation apparatus or past the latter and/or projected content is referred to, in particular, as an image that is registrable by the user. Properties of this image are referred to as image properties or image quality.

In particular, a controllable light source is a display device (e.g., a computer monitor, an LCD display, an OLED display or a 3-D monitor, etc.), a lamp, for example a surgical lamp or any other surgical light source, or an illumination of a surgical microscope or any other (robotic) visualization system. In particular, the controllable light source is arranged in an operating room. In this case, controllable should mean that, in particular, at least one property of the controllable light source can be altered, preferably over a plurality of levels or continuously, by means of an externally supplied control signal or control command. To this end, in particular, the controllable light source has an appropriately configured wired or wireless interface. The at least one controllable light source can also be part of the AR observation system.

The viewing direction sensor system of the AR observation apparatus can comprise both a line-of-sight sensor system and also, additionally or alternatively, a head viewing direction sensor system. By way of example, a line-of-sight sensor system comprises an eye/gaze tracking device. By way of example, such an eye/gaze tracking device is integrated in the AR observation apparatus, in particular in a pair of AR glasses. In the case of eye tracking (by means of an eye tracking sensor system), a viewing direction of each eye of the user is registered and/or determined. In the case of gaze tracking (likewise by means of an eye tracking sensor system), a point of fixation of the eyes (i.e., where the two eye tracking lines intersect) is determined. Eye tracking is implemented in image-based fashion in particular. To this end, at least one camera is directed at the eyes and images of the eyes are registered under weak infrared illumination. For evaluation purposes, the pupils of the eyes in the images are tracked by means of an algorithm and the line of sight for each of the eyes is determined, in particular estimated, therefrom. In this case, provision may be made for the eye/gaze tracking to have to be calibrated prior to a first application, within the scope of which a user must direct their gaze in a targeted fashion at calibration points projected into the AR observation apparatus. A head viewing direction sensor system in particular registers a relative position, i.e., an alignment and optionally an absolute position, of the AR observation apparatus, in particular the AR glasses, in relation to the environment, i.e., in particular, in relation to three-dimensional spatial coordinates of a coordinate system. As an alternative or in addition thereto, a change in the relative position is determined in relation to the environment. By way of example, the relative position can be determined by means of inside-out tracking, wherein a three-dimensional, geometric spatial model of the environment is created by means of an environment sensor system of the AR observation apparatus, in particular by means of a camera and/or depth sensors. By way of example, this is implemented by means of the simultaneous localization and mapping (SLAM) method, which is known per se. By means of the spatial model and inertial measurement units (IMUs) of the AR observation apparatus it is then possible to determine the relative position of the AR observation apparatus in the environment. Additionally, the controllable light sources can be registered and recognized by means of the environment sensor system of the AR observation apparatus such that the head viewing direction relative to a controllable light source can be determined. This can be implemented without markers or with the aid of markers arranged at the controllable light sources. Therefore, the viewing direction can be registered both in relation to a coordinate system of the environment and also, alternatively or additionally, relative to the AR observation apparatus.

In particular, the viewing direction sensor system generates a corresponding viewing direction signal and provides the latter to the control device. In this case, the viewing direction is determined continuously in particular such that a current viewing direction can be registered and/or determined and provided at all times.

The registered viewing direction in particular also comprises information as to whether the user gazes through the AR observation apparatus, in particular through a screen of the AR observation apparatus, or gazes past the AR observation apparatus, in particular past the screen, i.e., gazes directly at a region in the environment without the AR observation apparatus, in particular the screen, being arranged therebetween. By way of example, such looking past may occur if the user has a line of sight downward past the screen of the AR observation apparatus. Such information is considered when altering the at least one property of the at least one light source.

In particular, provision is made for the viewing direction to be registered or determined based on a line of sight registered by the line-of-sight sensor system and a head viewing direction registered by the head viewing direction sensor system, in particular by virtue of the registered line of sight being superimposed on the registered head viewing direction to form a viewing direction in relation to a coordinate system of the environment.

To evaluate the registered viewing direction the registered viewing direction is compared, for example, to directions and/or solid angles in the environment specified in relation to a relative position of the AR observation apparatus, at which specified directions and/or solid angles the at least one property of the at least one light source should be altered (as specified). In a simple example, provision can be made for, for example, a luminous intensity (or luminous power) of a controllable light source to always be regulated down if the evaluation yields that the registered viewing direction points in a direction specified to this end and/or a solid angle specified to this end. This can avoid or at least reduce the user being disturbed as a result of being dazzled.

The at least one property can be an electrical, an optical and/or a mechanical property of the at least one controllable light source. In particular, the at least one property is an optical property, that is to say the at least one property relates to light production and/or at least one light property of light produced by the controllable light source, for example luminous intensity, a power, a color distribution, a light temperature or a spectrum.

Altering the at least one property of the at least one controllable light source can be additionally implemented in consideration of a type of the controllable light source in particular. In particular, different properties can be altered in each case for different types of controllable light sources.

Altering the at least one property is implemented on the basis of the registered viewing direction. This means that the registered viewing direction is considered during the change, that is to say when selecting the at least one property and a scope of the change. In particular, provision can also be made for properties of a controllable light source to be altered differently if a registered viewing direction in respect of the environment is the same but the registered head viewing directions differ. In particular, this also allows cases to be considered in which the user does not gaze through the AR observation apparatus but past the latter.

Parts of the AR observation system, in particular the control device, can be embodied, either individually or together, as a combination of hardware and software, for example as program code that is executed on a microcontroller or microprocessor. However, provision can also be made for parts to be designed as application-specific integrated circuits (ASICs), either on their own or in combination.

In an embodiment, provision is made for the at least one property to be altered if the viewing direction is determined during the evaluation as being directed in the direction of a region in the environment lit or illuminated by the at least one controllable light source or if a viewing direction previously directed at such a region has departed from the latter again. As a result, the at least one property can be altered whenever the registered viewing direction is directed in the direction of a part of the at least one controllable light source relevant to eye adaptation, or whenever it departs from this relevant part. A lit region in the environment should denote, in particular, a region which emits light itself, for example a lamp or a display device. An illuminated region should denote, in particular, a region which does not emit light itself or which is not actively luminous, but in which there is an external excitation by light and the light is specularly and/or diffusely reflected by the region. To evaluate the registered viewing direction, a check is carried out as to whether or not the registered viewing direction is directed in the direction of the lit and/or illuminated regions of the at least one light source. In this case, the directions or relative positions of the lit and/or illuminated regions of the at least one light source are assumed to be known in particular. Further, a relative position of the AR observation apparatus in the environment is known in particular, and so it is possible to determine whether the registered viewing direction is directed in the direction of a lit and/or illuminated region of the at least one light source from the known relative positions of the lit and/or illuminated regions, the known relative position of the AR observation apparatus and the registered viewing direction. Should this be the case, the control device generates a control signal in order to alter the at least one property of the at least one light source.

In an embodiment, provision is made for the at least one property of the at least one controllable light source to be altered such that an image which is registrable by the user of the AR observation apparatus and which is of the region lit or illuminated by the at least one controllable light source satisfies at least one specified criterion. This allows image properties of the image registrable or registered by the user to be specified. In particular, what can be achieved thereby is that the image properties satisfy specified conditions. Even in the case of different and/or changing information sources (e.g., different lit and/or illuminated regions of different light sources in the environment), this allows the registration of these information sources to be designed uniformly such that the respectively registrable or registered image does not change so significantly even in the case of changing information sources that a smooth workflow is impeded because the eyes of the user have to adapt to the altered information sources during each change.

In a developing embodiment, provision is made for the at least one specified criterion to define a value range of at least one image parameter of the registrable image. As a result, the image parameters can be kept within the defined value range. In particular, an image parameter is a luminous intensity (irradiance or illuminance or power in relation to a solid angle), a brightness, a contrast ratio and/or at least one color property, for example a frequency spectrum or a specified color distribution. The at least one property of the at least one controllable light source is then altered in such a way that the image parameters (that is to say the image properties) of the image registrable and/or registered by the user of the lit and/or illuminated region remain within the specified value range. As a result, uniform registering by the eyes of the user can be facilitated even in the case of different information sources, which each correspond to different controllable light sources, without said eyes having to readapt every time the information source changes as a result of the change in the viewing direction. This assists a smooth workflow. By way of example, provision can be made for a luminous intensity of a controllable light source to be reduced when there is a change in the viewing direction from virtual content, which is projected into the display device of the AR observation apparatus, to the illuminated situs past the AR illumination apparatus. As a result of the reduction in the luminous intensity, the eyes of the user need not adapt in this case, and so the user can immediately register the illuminated situs without adaptation time and a smooth workflow is not impeded. To reduce the luminous intensity the control device generates a corresponding control signal and transmits the latter to the controllable light source. In particular, optimizing an image registrable by a user by means of the AR observation apparatus to a uniform and constant impression throughout in relation to brightness, contrast and/or color fidelity is facilitated as a result thereof.

In particular, provision can be made for the at least one criterion to be an optimization criterion which specifies at least one image property, in particular a value range of an image parameter, of an image registered by the user. Image properties registered by the user (e.g., a brightness, a contrast and/or a color distribution) or a registered image quality can be kept constant as a result.

In an embodiment, provision is made for an environment sensor system of the AR observation apparatus to be used to register a region of the environment that is registrable by the user, wherein lit or illuminated regions of the at least one controllable light source are identified based on sensor data that correspond to the registered region. As a result of this it is possible in automated fashion to define in particular directions and/or solid angles at which the at least one property of the at least one light source should be altered. By way of example, the environment sensor system can be a camera. In particular, the camera registers a region that is at least largely congruent with a maximum environment registrable by a user. Expressed differently, the environment sensor system, in particular the camera, preferably has the same field of view as the user such that it is possible to directly register what the user can register in the environment both through the AR observation apparatus and past the latter. As an alternative or in addition thereto, the environment sensor system can comprise a depth camera (e.g., time-of-flight, structural illumination, etc.). Based on sensor data that correspond to the registered region, lit or illuminated regions of the at least one controllable light source are identified. In this case, it is possible to carry out, by way of example, an object recognition by means of methods known per se, for example on the basis of computer vision and/or machine learning methods. The object recognition can be implemented without markers or with the aid of markers (e.g., Optinav markers, ArUco markers, etc.). The objects identified in the process are the controllable light sources, for example monitors, lamps, surgery lighting, etc. By way of a known relative position of the AR observation apparatus and the environment sensor system arranged thereon, it is possible to deduce the relative position of the recognized lit or illuminated regions of the controllable light sources. Based thereon it is possible to determine whether or not the registered viewing direction is directed in the direction of such a region.

In one embodiment provision is made for whether a region lit or illuminated by the at least one controllable light source is in the registered viewing direction to be determined in consideration of a three-dimensional model of the at least one controllable light source and/or the environment. This can improve the recognition of the lit and/or illuminated regions. By way of example, the three-dimensional model can be determined or may have been determined with the aid of the environment sensor system of the AR observation apparatus, for example by evaluating time-of-flight measurements from a depth camera. As an alternative or in addition thereto, the three-dimensional model can also be generated without registered measurement data, for example by stipulating a computer aided design (CAD) model which defines a shape and a relative position of objects, in particular of lit or illuminated regions of the at least one light source, in the environment. In this embodiment, use can be made of computer vision and/or machine learning methods, for example for recognizing lit and/or illuminated regions in consideration of or using the three-dimensional model.

To register the environment and controllable light sources arranged therein, it is additionally also possible to use at least one environment sensor system which is arranged in the environment and which has a known relative position, in addition to an environment sensor system of the AR observation apparatus.

In an embodiment, provision is made for at least one light property of light which is registrable by a user from a direction of the registered viewing direction and which emanates from the region lit or illuminated by the at least one controllable light source to be registered by means of the environment sensor system, wherein the change of the at least one property of the at least one light source is implemented in consideration of the registered at least one light property. As a result of this, it is possible to react directly to the light emanating from the lit or illuminated region. In particular, this facilitates closed-loop control of the at least one light property of the light emanating from the lit or illuminated region, by altering the at least one property of the associated controllable light sources. By way of example, the at least one light property can be a luminous intensity, a brightness, a contrast ratio and/or a color property, for example a color spectrum. Thus, provision can be made for a luminous intensity emanating from the lit or illuminated region or for a brightness registrable by a user, for example, to be registered. If, based on for example a specified value range for the luminous intensity and/or for the brightness, said luminous intensity and/or brightness is assessed as being too high or too low by the control device, the control device generates a control signal for reducing or increasing said luminous intensity and/or brightness and transmits said control signal to the at least one controllable light source which accordingly alters the luminous intensity and/or the brightness.

In an embodiment, provision is made for at least one light property of light which is registrable by a user from a direction of the registered viewing direction and which emanates from the region lit or illuminated by the at least one controllable light source to be determined on the basis of control data and/or state data of the at least one controllable light source, wherein the change of the at least one property of the at least one controllable light source is implemented in consideration of the determined at least one light property. As a result thereof, the at least one light property can be provided based on the control data and/or the state data of the at least one light source, even without registering the light by way of a sensor. Even if the light is registered by means of the environment sensor system, this embodiment offers advantages since, under certain circumstances, the at least one light property can be determined in improved fashion by means of the control data and/or the state data of the at least one controllable light source. By way of example, there can be a better estimate for or determination of a frequency spectrum of a computer monitor by way of the control data and/or the state data of the computer monitor than would usually be possible by means of an environment-registering camera on account of a distance. Overall, the at least one light property can therefore be determined in improved fashion. Further, known control data and/or state data of the control device facilitate an improved open-loop (or closed-loop) control of the at least one property of the at least one light source. The control data and/or state data are queried by means of the control device, in particular at the at least one light source, for example at an associated light source controller, and/or are transmitted from the at least one light source, for example the associated appliance controller, to the control device. By way of example, the at least one light property can likewise be a luminous intensity, a brightness, a contrast ratio and/or a color property, for example a color spectrum.

In an embodiment, provision is made for the at least one property of the at least one controllable light source to be additionally altered in consideration of at least one transmission property and/or absorption property of the AR observation apparatus. As a result, it is possible when altering the at least one property to consider a (frequency-dependent) attenuation of light which passes through a screen of the AR observation apparatus from the environment. By way of example, a color fidelity of an image displayed on a computer monitor can be maintained by considering the transmission properties and/or absorption properties, in particular a transmission spectrum and/or an absorption spectrum, by virtue of accordingly adapting a color distribution of the computer monitor in consideration of the transmission spectrum and/or the absorption spectrum such that changes in the registrable image caused by the transmission properties and/or absorption properties are compensated. The at least one transmission property and/or absorption property, in particular a transmission spectrum and/or absorption spectrum of the AR observation apparatus, can either be gathered from a datasheet of the AR observation apparatus or be determined empirically.

In an embodiment, provision is made for an opacity of the AR observation apparatus to be additionally altered at least in one portion of a field of view on the basis of the registered viewing direction. As a result, it is also possible to consider disturbances by non-controllable light sources. In particular, bright light sources in the field of view can be suppressed or attenuated such that the eyes of the user are not impeded. In particular, provision is therefore made for a determination of when the viewing direction is directed in the direction of a region in the environment lit or illuminated by at least one non-controllable light source. Should this be determined, the opacity of the AR observation apparatus is altered, in particular increased, at least in a portion of the field of view taken up by the non-controllable light source.

Further features relating to the configuration of the AR observation system arise from the description of configurations of the method. Here, the advantages of the AR observation system are respectively the same as in the configurations of the method.

### BRIEF DESCRIPTION OF THE DRAWINGS

The invention is explained in greater detail below on the basis of preferred exemplary embodiments with reference to the figures. In the figures:

FIG. 1 shows a schematic illustration of an embodiment of the augmented reality observation system for a surgical application; and

FIG. 2 shows a schematic flowchart of an exemplary embodiment of the method for operating an AR observation system during a surgical application.

### DETAILED DESCRIPTION

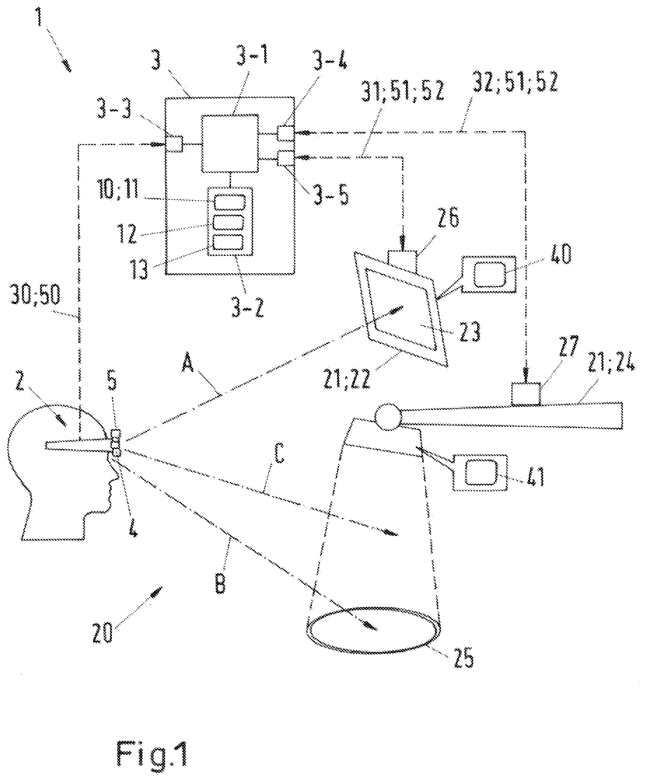

FIG. 1 shows a schematic illustration of an embodiment of the augmented reality observation system 1 for a surgical application. The AR observation system 1 is used in particular in the context of surgery on a patient in an operating room. In this case an environment 20 of the AR observation system 1 corresponds to a typical environment in an operating room, in particular. Two controllable light sources 21 are arranged in the environment 20 in exemplary fashion. One of the controllable light sources 21 is a display device 22 with a lit region 23. By way of example, the display device 22 can be a computer or appliance monitor or a freely positionable (3-D) visualization monitor of a robotic visualization system (not shown) used during surgery. The other controllable light source 21 is a lighting device 24 of a robotic visualization system (not shown) with an illuminated region 25. By way of example, the illuminated region 25 of the lighting device 24 coincides with a situs. The two controllable light sources 21 each have a light source controller 26, 27, by means of which at least one property of the controllable light sources 21 can be controlled in each case by stipulation of a corresponding control signal 31, 32.

The AR observation system 1 comprises an augmented reality observation apparatus 2 and a control device 3. The method for operating the augmented reality observation system 1 in a surgical application is described in more detail below on the basis of the augmented reality observation system 1.

The augmented reality observation apparatus 2 is configured as a pair of AR glasses and is worn on the head of a user during application. The augmented reality observation apparatus 2 comprises a viewing direction sensor system 4, which comprises a line-of-sight sensor system (not shown), for example an eye/gaze tracking device, and/or a head viewing direction sensor system (not shown), for example a head viewing direction sensor.

The control device 3 comprises a computing device 3-1 and a memory 3-2. The computing device 3-1 is configured to be able to carry out computational operations on data stored in the memory 3-2 and can, as a result thereof, carry out measures required to implement the method. By way of example, the computing device 3-1 comprises a microprocessor which for the purposes of carrying out parts of the method described in this disclosure can execute program code stored in the memory 3-2.

The viewing direction sensor system 4 of the AR observation apparatus 2 registers a viewing direction A, B, C of the user in the environment 20 by virtue of using the augmented reality observation system 1. In this case, the viewing direction A, B, C is determined and provided from the sensor data provided by the line-of-sight sensor system, for example by the eye/gaze tracking device, and by the head viewing direction sensor system, for example by the head viewing direction sensor. The registered viewing direction A, B, C is fed to the control device 3, for example as viewing direction signal 30, and is received for example by way of an interface 3-3 of the control device 3 configured to this end. By way of example, the viewing direction signal 30 comprises information regarding a direction and/or a solid angle which corresponds to the registered viewing direction A, B, C in relation to a relative position of the AR observation apparatus 2 or in relation to another coordinate system.

The control device 3 evaluates the registered viewing direction A, B, C. To this end, the registered viewing direction A, B, C is compared to for example specified directions and/or solid angles, in the case of which there should be a change in the at least one property 40, 41 of the at least one controllable light source 21.

If the registered viewing direction A, B, C is directed in such a specified direction and/or a specified solid angle, the control device 3 generates a control signal 31, 32 for altering at least one property 40, 41 of the at least one controllable light source 21. The type of the at least one property 40, 41 and a scope of change are linked to the specified direction and/or the specified solid angle, for example. By way of example, the specified directions and/or solid angles can be stored, together with the properties 40, 41 to be changed in each case, in a lookup table or database in the memory 3-2 of the control device 3 and can be queried from the latter when necessary. In a simple example, provision can be made for a luminous intensity of the at least one light source 21 to be reduced, in particular to a specified value, for specified directions and/or specified solid angles. The associated control signal 31, 32 is generated accordingly by the control device 3 and fed to the respective light source controller 26, 27 by way of interfaces 3-4, 3-5 configured to this end. The light source controller 26, 27 then alters the at least one property 40, 41 in accordance with the control signal 31, 32.

In particular, provision can be made for the at least one property 40, 41 to be altered if the viewing direction A, B, C is determined during the evaluation as being directed in the direction of a region 23 in the environment 20 lit, or a region 25 in the environment 20 illuminated, by the at least one controllable light source 21 or if a viewing direction A, B, C previously directed at such a region 23, 25 has departed from the latter again. To this end, the received viewing direction signal 30 is compared for example to solid angle ranges which, based on a relative position of the AR observation apparatus 2, coincide with the lit or illuminated regions 23, 25.

If it is determined by way of an evaluation result that the viewing direction A, B, C is directed in the direction of a region 23, 25 in the environment 20 lit or illuminated by the controllable light sources 21 then the at least one property 40, 41 of the controllable light source 21 corresponding to the region 23, 25 is altered by means of a control signal 31, 32 on the basis of the registered viewing direction A, B, C.

An example for the viewing direction A is the following: A user of the AR observation apparatus 2 gazes at displayed content of the display device 22, for example to register a live video data stream from a robotic visualization system (not shown) (i.e., there is no augmentation at this time). Since some of a luminous intensity emanating from the lit region 23 is absorbed by optical layers of the AR observation apparatus 2, an image registrable to this end by a user has a lower luminous intensity or a lower brightness behind the AR observation apparatus 2. To compensate this, the control device 3 generates a control signal 31, which increases a brightness of the display device 22 such that a luminous intensity is increased and the absorption by the optical layers of the AR observation apparatus 2 can be compensated, after determining that the viewing direction A is directed in the direction of the region 23 illuminated by the display device 22 through the AR observation apparatus 2.

An example for the viewing direction B is the following: During surgery, a user of the AR observation apparatus 2 gazes through the AR observation apparatus 2 at content of the display device 22 (i.e., there is no augmentation at this time) and carries out a surgical intervention with the aid of a robotic visualization system (not shown). To ensure an optimal illustration on the display device 22 by the robotic visualization system, the associated lighting device 24 is set to a high luminous intensity. To obtain a short, non-magnified view of the overall situs, the user briefly gazes directly at the overall situs in the illuminated region 25. This can be implemented by a head movement, with the user continuing to gaze through the AR observation apparatus 2, or by way of an eye movement by virtue of the user looking past the AR observation apparatus 2 at the bottom, corresponding to the viewing direction B illustrated in exemplary fashion. Then, it is determined for the registered viewing direction B that the viewing direction B is directed past the AR observation apparatus 2 at the bottom and directly on the illuminated region 25 of the lighting device 24, that is to say the overall situs. Since the high luminous intensity emanating from the illuminated region 25 would dazzle the user and an adaptation of the eyes would be required, the control device 3, following the determination that the registered viewing direction B is directed in the direction of the illuminated region 25, generates a control signal 32 which reduces the luminous intensity of the lighting device 24 such that the user is no longer dazzled and can register the overall situs in the illuminated region 25 without interruption or impediment. If the user subsequently gazes on the display device 22 again, in order to register the situs magnified by means of the robotic visualization system once again, the control device 3 once again generates a control signal 32 which prompts the lighting device 24 to increase the luminous intensity back to the previous value in order to ensure optimal illumination during the registration by means of the robotic visualization system.

By contrast, for the viewing direction C there is no change in properties of the controllable light sources 21 since it is directed in neither of the two regions 23, 25.

Provision can be made for the at least one property 40, 41 of the at least one controllable light source 21 to be altered such that an image which is registrable by the user of the AR observation apparatus 2 and which is of the region 23, 25 lit or illuminated by the at least one controllable light source 21 satisfies at least one specified criterion 10. As a result, images registered by the user from different information sources (environment, controllable light sources, AR content, etc.) via and/or through the AR observation apparatus 2 can be altered and more particularly optimized in view of a uniform impression. By way of example, the registrable image can be estimated on the basis of optical properties (transmission properties, reproduction properties during the reproduction of AR content, etc.) of the AR observation apparatus 2. In this case, the control device 3 checks whether or not the at least one criterion 10 is satisfied, and alters the at least one property 40, 41 of the at least one controllable light source 21 until the at least one criterion 10 is satisfied.

In a development, provision can be made, in particular, for the at least one criterion 10 to define a value range 11 of at least one image parameter of the registrable image. By way of example, such an image parameter can be a luminous intensity, a brightness, a contrast ratio and/or a color property, for example a color spectrum.

Provision can be made for the AR observation apparatus 2 to comprise an environment sensor system 5, for example a camera that registers the environment 20. An environment sensor system 5 can also be a depth sensor which registers and provides a three-dimensional image representation of the environment 20. The environment sensor system 5 is used to register a region of the environment 20 that is registrable by the user, wherein lit or illuminated regions 23, 25 of the at least one controllable light source 21 are identified based on sensor data that correspond to the registered region. In the process, use can be made, for example, of an object recognition by means of methods of computer vision and/or machine learning, in order to recognize the controllable light sources 21 and/or the associated regions 23, 25. In the process, provision can be made for the use of markers on the controllable light sources 21 in order to assist the recognition.

Provision can be made for whether a region 23 lit, or a region 25 illuminated, by the at least one controllable light source 21 is in the registered viewing direction A, B, C to be determined in consideration of a three-dimensional model 12 of the at least one controllable light source 21 and/or the environment 20. The AR observation apparatus 2 and the registered viewing direction A, B, C can be located within the three-dimensional model 12 such that, proceeding therefrom, it is possible to determine the direction in which the registered viewing direction A, B, C is directed. In this case, use can be made of computer vision and/or machine learning, for example for recognizing the lit and/or illuminated regions 23, 25 in consideration of or using the three-dimensional model 12.

Provision can be made for at least one light property 50 of light which is registrable by a user from a direction of the registered viewing direction A, B, C and which emanates from the region 23 lit, or region 25 illuminated, by the at least one controllable light source 21 to be registered by means of the environment sensor system 5, for example by means of a camera, wherein the change of the at least one property 40, 41 of the at least one light source 21 is implemented in consideration of the registered at least one light property 50. The registered at least one light property 50 can comprise a luminous intensity or brightness, a contrast ratio or a color or frequency spectrum, for example. The registered at least one light property 50 is fed to the control device 3 by the AR observation apparatus 2. Based on the registered at least one light property 50, the control device 3 determines the at least one property 40, 41 of the controllable light source 21 that should be altered, and the scope of the change. In the examples for viewing directions A and B, described above, the environment sensor system 5 can register, for example, a luminous intensity (or brightness) emanating from the regions 23, 25 as a light property 50, wherein the control device 3 controls or regulates the luminous intensity (or the brightness) of the display device 22 or of the lighting device 24 to a respectively required value proceeding therefrom.

Provision can be made for at least one light property 50 of light which is registrable by a user from a direction of the registered viewing direction A, B, C and which emanates from the region 23 lit, or region 25 illuminated, by the at least one controllable light source 21 to be determined on the basis of control data 51 and/or state data 52 of the at least one controllable light source 21, wherein the change of the at least one property 40, 41 of the at least one controllable light source 21 is implemented in consideration of the determined at least one light property 50. To this end, the control device 3 queries the control data 51 and/or state data 52 at the light source controllers 26, 27 via the interfaces 3-4, 3-5 and takes these into account when generating the control signals 31, 32. The control data 51 and/or state data 52 of the display device 22 can comprise a brightness, a contrast ratio or a color setting, for example. The control data 51 and/or state data 52 of the lighting device 24 can comprise a luminous intensity, for example.

Provision can be made for the at least one property 40, 41 of the at least one controllable light source 21 to be additionally altered in consideration of at least one transmission property 13 and/or absorption property of the AR observation apparatus 2. By way of example, the at least one transmission property 13 comprises a transmission spectrum which describes a frequency-dependent or wavelength-dependent transmission of the AR observation apparatus 2, in particular of a screen of the AR observation apparatus 2. With the aid of the transmission spectrum it is possible to estimate an image of the environment and, in particular, of the controllable light sources 21 registrable by a user, and so for example the at least one property 40 of the display device 22 can be altered in such a way that the frequency-dependent or wavelength-dependent absorption of the AR observation apparatus 2 can be compensated for. As a result, it is possible in particular to maintain a color fidelity of content displayed on the display device 22. By way of example, the at least one transmission property 13 and/or absorption property is taken from a datasheet of the AR observation apparatus 2 or determined empirically, and stored in the memory 3-2 of the control device 3.

Provision can be made for an opacity of the AR observation apparatus 2 to be additionally altered at least in one portion of a field of view on the basis of the registered viewing direction A, B, C. This allows light from non-controllable light sources (not shown) to be suppressed. By way of example, if a door to a darkened operating room is opened, disturbing light can reach the operating room from the outside and can disturb the user of the AR observation apparatus 2 in their workflow. If the viewing direction A, B, C is directed in the direction of the non-controllable light source, a position, corresponding herewith, of a screen of the AR observation apparatus 2 that is changeable in respect of its opacity is darkened such that the light of the non-controllable light source is absorbed more strongly than light reaching the AR observation apparatus 2 from surrounding regions of the environment 20.

With the aid of the AR observation system 1 for a surgical application described in this disclosure and of the method for operating the AR observation system 1 in a surgical application, it is possible to reduce or even prevent an impediment to the workflow of a user of an AR observation apparatus 2 by light sources in the environment of the surgical application.

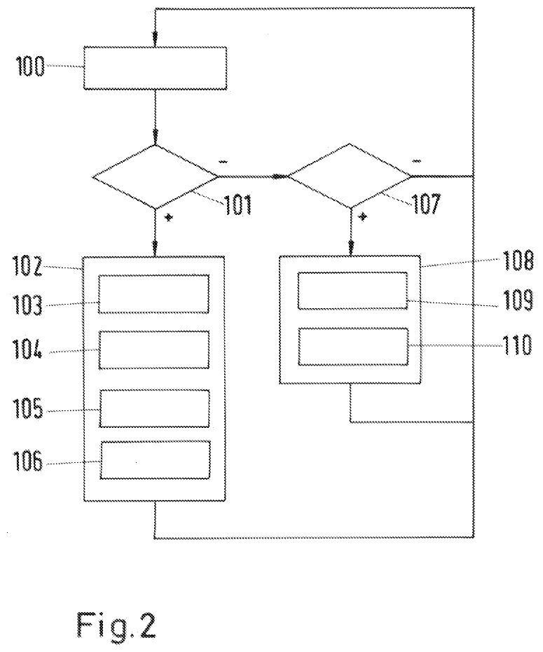

Shown in FIG. 2 is a schematic flowchart of an exemplary embodiment of the method for operating an AR observation system during a surgical application.

In a measure 100, a viewing direction of a user is registered by means of a viewing direction sensor system of an AR observation apparatus of the AR observation system. In the process, both a line of sight and a head viewing direction are registered in particular, and so it is possible to also determine a relative viewing direction of the eyes in relation to the head. Therefore, the registered viewing direction in particular also comprises information in respect of whether a user of the AR observation apparatus gazes through or gazes past the latter.

In a measure 101, the registered viewing direction is evaluated. To this end, a check is carried out as to whether the registered viewing direction is directed in the direction of a lit or illuminated region of at least one controllable light source in the environment.

Should this be the case, a measure 102 is carried out. The measure 102 comprises measures 103 to 106. In measure 103, a luminous intensity of light emanating from the lit or illuminated region is registered by means of an environment sensor system, in particular by means of a camera, of the AR observation apparatus. The extent to which the registered luminous intensity deviates from a target value specified as a specified criterion, in particular, is determined in measure 104. In this case, the specified target value corresponds to an optimal brightness of an image which is registered by a user of the AR observation apparatus and which is of the controllable light sources in the environment. In measure 105, a control signal corresponding to the target value is generated by means of the control device and fed to the controllable light source. In measure 106, the luminous intensity of the controllable light source is altered in accordance with the control signal such that it corresponds to the target value for the luminous intensity.

By contrast, if an evaluation result in measure 101 yields that the viewing direction is not directed in the direction of a lit or illuminated region, a check is carried out in a measure 107 as to whether the registered viewing direction had previously been directed in the direction of such a region. Measure 108, which comprises measures 109 and 110, is carried out should this be the case. In measure 109, a control signal corresponding to the original value of the luminous intensity of the controllable light source is generated by means of the control device and fed to the controllable light source. In measure 110, the luminous intensity of the controllable light source is altered in accordance with the control signal such that it corresponds to the original value for the luminous intensity again.

The method is repeated cyclically such that a smooth workflow with an always optimal light intensity of the controllable light sources in the environment of the AR observation apparatus is facilitated.

The shown embodiments are merely exemplary. In particular, it is also possible to alter different properties of the at least one controllable light source to the properties described and, in particular, a plurality of properties of the at least one controllable light source.

### LIST OF REFERENCE SIGNS

1 Augmented reality (AR) observation system
2 Augmented reality (AR) observation apparatus
3 Control device
3-1 Computing device
3-2 Memory
3-3 Interface
3-4 Interface
3-5 Interface
4 Viewing direction sensor system
5 Environment sensor system
10 Specified criterion
11 Value range (image parameters)
12 Three-dimensional model
13 Transmission property
20 Environment
21 Controllable light source
22 Display device
23 Lit region
24 Lighting device
25 Illuminated region
26 Light source controller
27 Light source controller
30 Viewing direction signal
31 Control signal
32 Control signal
40 Property
41 Property
50 Light property
51 Control data
52 State data
A, B, C Viewing direction
100-110 Method measures

1. A method for operating an augmented reality observation system in a surgical application, wherein a viewing direction of a user is registered by means of a viewing direction sensor system of an AR observation apparatus,
wherein the registered viewing direction is evaluated by means of a control device, and
wherein at least one property of at least one controllable light source in the environment is altered by means of the control device by means of a control signal on the basis of the registered viewing direction.

2. The method as claimed in claim 1, wherein the at least one property is altered if the viewing direction is determined during the evaluation as being directed in the direction of a region in the environment lit or illuminated by the at least one controllable light source or if a viewing direction previously directed at such a region has departed from the latter again.

3. The method as claimed in claim 1, wherein the at least one property of the at least one controllable light source is altered such that an image which is registrable by the user of the AR observation apparatus and which is of the region lit or illuminated by the at least one controllable light source satisfies at least one specified criterion.

4. The method as claimed in claim 3, wherein the at least one specified criterion defines a value range of at least one image parameter of the registrable image.

5. The method as claimed in claim 2, wherein an environment sensor system of the AR observation apparatus is used to register a region of the environment that is registrable by the user, wherein lit or illuminated regions of the at least one controllable light source are identified based on sensor data that correspond to the registered region.

6. The method as claimed in claim 2, wherein whether a region lit or illuminated by the at least one controllable light source is in the registered viewing direction is determined in consideration of a three-dimensional model of the at least one controllable light source and/or the environment.

7. The method as claimed in claim 2, wherein at least one light property of light which is registrable by a user from a direction of the registered viewing direction and which emanates from the region lit or illuminated by the at least one controllable light source is registered by means of the environment sensor system, wherein the change of the at least one property of the at least one light source is implemented in consideration of the registered at least one light property.

8. The method as claimed in claim 2, wherein at least one light property of light which is registrable by a user from a direction of the registered viewing direction and which emanates from the region lit or illuminated by the at least one controllable light source is determined on the basis of control data and/or state data of the at least one controllable light source, wherein the change of the at least one property of the at least one controllable light source is implemented in consideration of the determined at least one light property.

9. The method as claimed in claim 1, wherein the at least one property of the at least one controllable light source is additionally altered in consideration of at least one transmission property of the AR observation apparatus.

10. An augmented reality observation system for a surgical application, comprising:
an augmented reality observation apparatus with a viewing direction sensor system,
wherein the viewing direction sensor system is configured to register a viewing direction of a user of the AR observation apparatus, and
a control device,
wherein the control device is configured to evaluate the registered viewing direction and to alter at least one property of at least one controllable light source in the environment on the basis of the registered viewing direction by means of a control signal.
